(19) United States
(12) Patent Application Publication
(10) Pub. No.: US 2004/0005600 A1
(43) Pub. Date: Jan. 8, 2004

(54) Method of designing synthetic nucleic acid sequences for optimal protein expression in a host cell

(76) Inventors: Evelina Angov, Bethesda, MD (US); Jeffrey A. Lyon, Silver Spring, MD (US); Randall L. Kincaid, Potomac, MD (US)

Correspondence Address: ATTN: MCMR-JA (Ms. Elizabeth Arwine- PATENT ATTY), U. S. Army Medical Research and Materiel Command, 504 Scott Street, Fort Detrick, MD, 21702-5012, US

(21) Appl. No.: 10/404,668

(22) Filed: Apr. 1, 2003

Related U.S. Application Data
(60) Provisional application No. 60/369,741, filed on Apr. 1, 2002.
(60) Provisional application No. 60/379,688, filed on May 9, 2002.
(60) Provisional application No. 60/425,719, filed on Nov. 12, 2002.

(51) Int. Cl. C12Q 1/68; C07H 21/04; C12N 1/21

(52) U.S. Cl. 435/6; 536/23.7; 435/252.33

(57) ABSTRACT

The present invention provides a method for modifying a wild type nucleic acid sequence encoding a polypeptide to enhance expression and accumulation of the polypeptide in the host cell by harmonizing synonymous codon usage frequency between the foreign DNA and the host cell DNA. This can be done by substituting codons in the foreign coding sequence with codons of similar usage frequency from the host DNA/RNA which code for the same amino acid. The present invention also provides novel synthetic nucleic acid sequences prepared by the method of the invention.

This application claims the benefit of priority from an earlier filed provisional application serial No. 60/369,741 filed on Apr. 1, 2002 and provisional application serial No. 60/379,688 filed on May 9, 2002, and provisional application 60/425,719 filed on Nov. 12, 2002.

### FIELD OF THE INVENTION

This invention generally relates to genetic engineering and more particularly to methods for designing a synthetic gene de novo for the optimal expression of a known protein coding sequence in a host cell and further to increasing solubility and biological activity of the expressed protein.

### BACKGROUND OF THE INVENTION

One of the primary goals of biotechnology is to provide large amounts of a desired protein by expressing a foreign gene in a host cell, for example E. coli. Significant advances have been made in pursuit of this goal, but the expression of some foreign genes in host cells remains problematic. Numerous factors are involved in determining the ultimate level and biological activity of a protein produced from expressing a foreign gene in a host cell. Among them are toxicity of the gene product and consequent instability of the foreign DNA sequence, level of RNA produced, improper or inefficient translation of the RNA, improper folding or insolubility of the translated protein and difficulties in isolating the protein from the cell.

Various nucleotide sequences affect the expression levels of protein encoded by a foreign DNA sequence introduced into a cell. These include the promoter sequence, the structural coding sequence that encodes the desired foreign protein, 3′ untranslated sequences, and polyadenylation sites. Because the structural coding region introduced into the cell is often the only “non-host” sequence introduced, it has been suggested that it could be a significant factor affecting the level of expression of the protein. This problem is created by the degeneracy of the genetic code and the fact that the various tRNA isoacceptors are not all used at the same frequencies by a single organism and the usage pattern varies from species to species as shown in Table 1. As illustrated in this table, the frequency with which synonymous codons (those specifying the same amino acid) are used in an organism is not simply an arithmetic average (e.g., 25% in the case where four codons specify an amino acid such as valine). Rather, there are clear biases in the codon usage frequency in a given organism, and these biases can vary dramatically between different organisms. Although the fundamental code for protein translation remains the same, it appears as though significant divergence has occurred in how synonymous codons are used, analogous to a language having evolved distinct dialects.

TABLE 1
Codon Usage Frequency for Three Species

| | | | | | | | Codon Usage | | |
|---|---|---|---|---|---|---|---|---|---|
| | | Codon Usage Frequency | | | | | Frequency | | |
| | AA | E. | P. | | | AA | E. | P. | |
| codon | Residue | coil | falciparum | Human | codon | Residue | coil | falciparum | Human |
| GCA | Ala | 0.28 | 0.43 | 0.13 | CTA | Leu | 0.00 | 0.08 | 0.03 |
| GCC | Ala | 0.10 | 0.11 | 0.53 | CTC | Leu | 0.07 | 0.02 | 0.26 |
| GCG | Ala | 0.26 | 0.06 | 0.17 | CTG | Leu | 0.83 | 0.02 | 0.58 |
| GCT | Ala | 0.35 | 0.40 | 0.17 | CTT | Leu | 0.04 | 0.11 | 0.05 |
| AGA | Arg | 0.00 | 0.59 | 0.10 | TTA | Leu | 0.02 | 0.63 | 0.02 |
| AUG | Arg | 0.00 | 0.17 | 0.10 | TTG | Leu | 0.03 | 0.14 | 0.06 |
| CGA | Arg | 0.01 | 0.09 | 0.06 | AAA | Lys | 0.74 | 0.81 | 0.18 |
| CGC | Arg | 0.25 | 0.02 | 0.37 | AAG | Lys | 0.26 | 0.19 | 0.82 |
| CGG | Arg | 0.00 | 0.01 | 0.21 | ATG | Met | 1.00 | 1.00 | 1.00 |
| CGT | Arg | 0.74 | 0.12 | 0.07 | TTC | Phe | 0.76 | 0.16 | 0.80 |
| AAC | Asn | 0.94 | 0.14 | 0.78 | TTT | Phe | 0.24 | 0.84 | 0.20 |
| AAT | Asn | 0.06 | 0.86 | 0.22 | CCA | Pro | 0.15 | 0.44 | 0.16 |
| GAC | Asp | 0.67 | 0.13 | 0.75 | CCC | Pro | 0.00 | 0.11 | 0.48 |
| GAT | Asp | 0.33 | 0.87 | 0.25 | CCG | Pro | 0.77 | 0.05 | 0.17 |
| TGC | Cys | 0.51 | 0.14 | 0.68 | CCT | Pro | 0.08 | 0.40 | 0.19 |
| TGT | Cys | 0.49 | 0.86 | 0.32 | AGC | Ser | 0.20 | 0.06 | 0.34 |
| CAA | Gln | 0.14 | 0.87 | 0.12 | AGT | Ser | 0.03 | 0.32 | 0.10 |
| CAG | Gln | 0.86 | 0.13 | 0.88 | TCA | Ser | 0.02 | 0.26 | 0.05 |
| GAG | Glu | 0.78 | 0.85 | 0.25 | TCC | Ser | 0.37 | 0.08 | 0.28 |
| GAG | Glu | 0.22 | 0.15 | 0.75 | TCG | Ser | 0.04 | 0.05 | 0.09 |
| GGA | Gly | 0.00 | 0.44 | 0.14 | TCT | Ser | 0.34 | 0.23 | 0.13 |
| GGC | Gly | 0.38 | 0.05 | 0.50 | ACA | Thr | 0.04 | 0.54 | 0.14 |
| GGG | Gly | 0.02 | 0.10 | 0.24 | ACC | Thr | 0.55 | 0.12 | 0.57 |
| GGT | Gly | 0.59 | 0.42 | 0.12 | ACG | Thr | 0.07 | 0.10 | 0.15 |
| CAC | His | 0.83 | 0.15 | 0.79 | ACT | Thr | 0.35 | 0.25 | 0.14 |
| CAT | His | 0.17 | 0.85 | 0.21 | TGG | Trp | 1.00 | 1.00 | 1.00 |
| ATA | Ile | 0.00 | 0.56 | 0.05 | TAC | Tyr | 0.75 | 0.11 | 0.74 |
| ATC | Ile | 0.83 | 0.07 | 0.77 | TAT | Tyr | 0.25 | 0.89 | 0.26 |
| ATT | Ile | 0.17 | 0.37 | 0.18 | UTA | Val | 0.26 | 0.41 | 0.05 |
| | | | | | GTC | Val | 0.07 | 0.06 | 0.25 |
| | | | | | GTG | Val | 0.16 | 0.14 | 0.64 |
| | | | | | GTT | Val | 0.51 | 0.39 | 0.07 |

| | |
|---|---|
| Eschericia coil | Data Reference Set, Volume 3: Data Files, |
| | Genetics Computer Group, Sequence Analysis Software Package |
| | http://www.kazusa.or.jp/codon/P.html; select |
| P. falciparum: | Plasmodium falciparum |
| Homo sapiens. | http://bioinformatics.weizmann.ac.il/databases/codon/hum.cod |

E. coli expression of some Plasmodium falciparum protein antigens has been difficult owing to the strong bias toward A/T synonymous codon usage by this parasite (see Table 1). Problems that have been encountered include poor protein expression, expression of insoluble protein, and plasmid instability. A/T rich codons are used infrequently in E. coli, which is thought to contribute to problems with heterologous expression of P. falciparum genes in this host. In the past, researchers have attempted to improve heterologous protein expression for many species by applying the principle of “codon optimization”, which is to substitute frequently used E. coli codons, synonymously, for the infrequently used codons specified by the foreign gene. In this approach, the same E. coli codon is used every time a given amino acid is specified (e.g., CGG for every arginine)

However, more likely, expression problems occur because expression and formation of secondary structure of nascent protein occur co-translationally and depend on the rate of ribosome progression through different regions of the mRNA. This rate of ribosome progression is thought to depend upon the codon frequency, which may be related directly to t-RNA isoacceptors abundance (Ikemura, T., 1981, J.Mol. Biol. 151, 389-409). Thus, frequently used codons are translated quickly and infrequently used codons are translated slowly. Regions of coding sequence with slower translation rates may contain clusters of infrequently used codons and appear to be associated with unstructured intradomain segments in the protein that separate defined domain structures such as alpha helices and beta-pleated sheets. Temporary ribosomal “pausing” on the intradomain segment is thought to allow the preceding nacent protein domain to complete folding prior to continuing synthesis of the next domain (Thanaraj, T A & Argos, P., 1996, Protein Sci. 5:1594-1612). The selection of codons at each position in an amino acid sequence may indeed reflect a purposeful evolutionary adaptation that defines temporal requirements for proper protein folding. Thus, incorrect protein folding is likely to occur when a heterologous gene is characterized by codon usage patterns that are disharmonious with the t-RNA abundances of the expression host. A strategy to overcome this problem is to make synthetic genes having codon usage patterns that are “harmonized” to those of the expression host. The goal of codon harmonization, then, is to deduce the relative rate of translation at each position in the foreign protein's sequence, based on the frequency with which its codon is used by that organism, and then match that rate to the rate anticipated for a synonymous codon in the host (E. coli) that has a corresponding frequency of usage. This concept is very different from that of codon optimization, wherein the rate of codon translation at each amino acid is designed to be high (optimized) and thus cannot be altered through selective recruitment of less frequently used t-RNA populations.

One can also expect that this approach would be useful for insuring optimal E. coli expression of proteins from species other than Plasmodia, as well as for insuring the optimal expression of foreign genes in species other than E. coli.

### SUMMARY OF THE INVENTION

Briefly, a method for modifying a nucleotide sequence for enhanced accumulation and biological activity of its protein or polypeptide product in a host cell is provided. In addition, a method for the design of synthetic genes, de novo, for enhanced accumulation and biological activity of its encoded protein or polypeptide product in a host cell is provided.

Surprisingly, it has been found that, by using the concept of codon harmonization, partially modified as well as completely synthetic P. falciparum antigen genes give dramatic improvements in the yield of soluble, and likely correctly folded, protein. The method of the present invention is valuable for producing large amounts of a protein, e.g. a vaccine candidate that heretofore may have been unavailable for testing because of low expression, for producing pharmaceutically valuable recombinant proteins such as growth factors, or other medically useful proteins, and for producing reagents that may enable dramatic advances in drug discovery research and basic proteomic research.

Thus, the present invention is drawn to a method for modifying structural coding sequence encoding a polypeptide to enhance accumulation of the polypeptide in a host cell, which comprises determining the amino acid sequence of the polypeptide encoded by the structural coding sequence and harmonizing codon frequency between the foreign DNA/RNA and the host cell DNA/RNA. This can be done by substituting codons in the foreign coding sequence with codons of similar frequency from the host DNA/RNA which code for the same amino acid. Therefore, the result would be the same amino acid sequence of the foreign gene encoded by host cell codons chosen on the basis of codon frequency.

The present invention is further directed to synthetic structural coding sequences produced by the method of this invention where the synthetic coding sequence expresses its protein product in host cells at levels significantly higher than corresponding wild-type coding sequences.

The present invention is also directed to a novel method for designing a synthetic gene for optimal expression of the encoded protein comprising determination of the frequency of usage of foreign gene codons and frequency of usage of host codons and substituting the foreign codons with a more-preferred host codon of similar frequency of usage, while maintaining a structural gene encoding the polypeptide, wherein these steps are performed sequentially and have a cumulative effect resulting in a nucleotide sequence containing a preferential utilization of the host cell codons for foreign codons for one or more of the amino acids present in the polypeptide.

The present invention is also directed to a method which further includes a systematic bioinformatic analysis of secondary and tertiary structure of the protein sequence to be expressed that is carried out to correlate the utilization of infrequently-used codons with regions of protein structure (including but not limited to “turns” at the ends of coils, anti-parallel strands, extended beta sheets or helices and regions of disordered structure) that might necessarily require time to fold properly. Additional bioinformatic information such as protein sequence homology, motif homologies and secondary and/or tertiary structure homologies may be “overlaid” to refine the anticipated need for inclusion or exclusion of such codons. Furthermore, bioinformatic evaluation and design of nucleic acid sequence may be carried out to minimize formation of self-annealing hybrid (“stem-loop”) structures in the resulting mRNA transcript that could affect translational rate, independent of frequency of codon usage.

The present invention is further directed to host cells containing synthetic nucleic acid sequence(s), e.g. DNA or RNA, prepared by the methods of this invention and the expressed product of said synthetic sequence.

Therefore, it is an object of the present invention to provide synthetic DNA/RNA sequences that are capable of expressing their respective proteins at relatively higher levels and/or with higher biological activity than the corresponding wild-type sequence and methods for the preparation of such sequences, which may include computational algorithms, software for prediction and validation of properly harmonized synthetic gene sequences.

It is also an object of the present invention to provide a method for improving protein accumulation from a foreign gene transformed into a host cell and/or improving the solubility of said protein, by designing a harmonized synthetic gene, by determining the frequency of occurrence of foreign gene codons and host codons, and substituting the nucleotide sequence of the foreign gene with host codons of similar frequency.

### BRIEF DESCRIPTION OF THE DRAWINGS

FIGS. 1A, 1B, 1C, 1E and 1E. Example of spreadsheets from Excel program applied for harmonization of P. falciparum and E. coli. 1A) FVO wild-type codons. 1B) proposed codons. 1C) Codon Frequency Reference Values,Columns A-H. 1D) Codon Frequency Reference Values, Columns I-Q. 1E) Harmonize.

FIG. 2. Soluble Expression of LSA-NRC from Tuner(DE3) containing plasmids pETKLSA-NRC/E or pETKLSA-NRC/H. Lanes1-4 pETK LSA-NRC/E, containing an lsa-nrc/E gene whose codons were “optimized” for E. coli expression by selection of the most common codon for each amino acid. Lanes 5-8 pETK LSA-NRC/H, containing an lsa-nrc/H gene with codons “harmonized” for E. coli expression by selection of codons that allowed the rate of translation to more closely match that predicted for genes being translated in P. falciparum. Lanes 1, 2, 5, 6 are stained SDS-PAGE gels; Lanes 3, 4, 7, 8 are Western blots of equivalent gels; Uninduced expression sample lanes 1, 3, 5, 7: induced (0.5 mM IPTG) sample lanes 2, 4, 6, 8. Lane M: pre-stained markers. Molecular weights are given on the left×10⁻³.

FIG. 3. Coomassie blue stained SDS-PAGE for partially purified wild type MSP-142 (FVO) vs. single site pause mutant (FMP003).

FIG. 4. Coomassie stained SDA-PAGE on partially purified MSP-42 (FVO) (Wild-type vs. Single site pause mutant (FMP003) vs. Initiation Complex harmonized (FMP007).

FIGS. 5A and 5B. A) Coomassie blue stained SDS-PAGE (left panel) and Western blot analysis (right panel) of lysates from bacteria expressing FMP003, FMP007, or full gene harmonized. B) Solubility and partial purification of full gene harmonized MSP142 (FVO) in the presence (+Tween 80) and absence (−Tween 80) of Tween 80 detergent.

### DETAILED DESCRIPTION

The following definitions are provided for clarity of the terms used in the description of this invention.

Foreign gene. A nucleic acid which is not part of the host cell genome.

Synthetic gene. A nucleic acid which has been modified from its wild-type sequence.

Host cell. A cell into which a foreign gene is introduced. The host cell can be prokaryotic or eukaryotic.

It has been discovered that a nucleotide sequence capable of enhanced expression in host cells can be obtained by harmonizing the frequency of codon usage in the foreign gene at each codon in the coding sequence to that used by the host cell.

Therefore, the present invention provides a method for modifying a nucleic acid sequence encoding a polypeptide to enhance expression and accumulation of the polypeptide in the host cell. In another aspect, the present invention provides novel synthetic nucleic acid sequences, encoding a polypeptide or protein that is foreign to a host cell, that is expressed at greater levels and with greater biological activity than in the host cell as compared to the wild-type sequence if expressed in the same host cell.

The invention will primarily be described with respect to the preparation of synthetic DNA sequences (also referred to as nucleotide sequences, structural coding sequences or genes) which encode the P. falciparum genes, but it should be understood that the method of the present invention is applicable to any coding sequence encoding a protein foreign to a host cell in which the protein is expressed.

DNA sequences modified by the method of the present invention are effectively expressed at a greater level in host cells than the corresponding non-modified DNA sequence. In accordance with the present invention, DNA sequences are modified to harmonize codon usage in the foreign gene with codon usage in the host cell by substituting synonymous codons from the host cell for foreign gene codons of similar usage frequency, where necessary. In the first analysis, codons that will be changed are those that are used more frequently in the host cell than in the foreign gene. Those foreign gene codons will be replaced with synonymous host cell codons that are used at the same frequency or less frequently. In the second analysis, after overlaying bioinformatics approaches, the decision to actually change a codon will depend on the location of the amino acid in the polypeptide. For example, all codons that are associated with intradomain segments will be replaced according to the paradigm described above. For codons associated with domains, it is probably sufficient to replace the codon only if the codon usage frequencies vary by +/−50%. Depending on the degree of similarity of codon usage preferences in the foreign gene and the host cell, this could produce various results, ranging from no or little modification of the DNA sequence to many modifications. The former outcome would be expected for situations where the foreign gene and the expression host have relatively similar codon usage preferences or where bioinformatics focuses attention onto the coding sequences of the intradomain segments. The latter outcome would be expected for situations where the foreign gene and the expression hosts have extremely different codon usage preferences. In either case it would be expected that the minimum number of changes required would be those that harmonize codon usage within the intradomain segments and especially those intradomain segments associated with the initiation complex. It should be understood that heterologous expression of proteins may involve additional unknown complexities, in addition to a need for harmonized sequence. It would be anticipated that iterative, empirical tests of harmonized sequence may be needed to obtain optimal expression.

The following description presents one process by which codon usage frequencies between genes can be compared. The present process was designed using a commercially available Excel program. Any program which supports a relational database which supports a set of operations defined by relational algebra can be used or designed. It generally includes tables composed of columns and rows for the data contained in the database. Each table has a primary key, being any column or set of columns the values of which uniquely identify the rows in the table. The relational database is subject to a set of operations (select, project, product, join, and divide) which form the basis of the relational algebra governing relations within the database. Relational databases are well known and documented (see, e.g., Nath, A. The Guide To SQL Server, 2^{nd} ed. Addison-Wesley Publishing Co., 1995 (which is incorporated herein by reference for all purposes). The amino acid sequence of the protein can be analyzed using commercially available computer software such as the “BackTranslate” program of the GCG Sequence Analysis Software Package, DNA Star, Vector NTI, or a simple “lookup table” written in Excel, or a modification of a commercial package. A computer program product including a computer-usable medium having computer-readable program code embodied thereon relating to comparing codon frequencies and translation rate is envisioned. The computer program product includes computer-readable program code for providing, within a computing system, an interface for receiving a selection of one or more target gene sequence, determining codon frequencies of said target gene and comparing to frequencies of selected host gene sequence, determining whether or not a codon should be modified to match a host codon, and displaying the results of the determination.

In the process used in the Examples below, a text file is created that contains the entire wild type target gene sequence of the protein of interest, such that each codon is on a separate line separated by a hard return.

This text file is imported into Excel simply by opening the file with Excel. Each codon of the sequence should occupy a single cell and all codons should be held in a single column of the spreadsheet. Alternatively, codons can be entired from the keyboard, one codon per cell all codons in a single column.

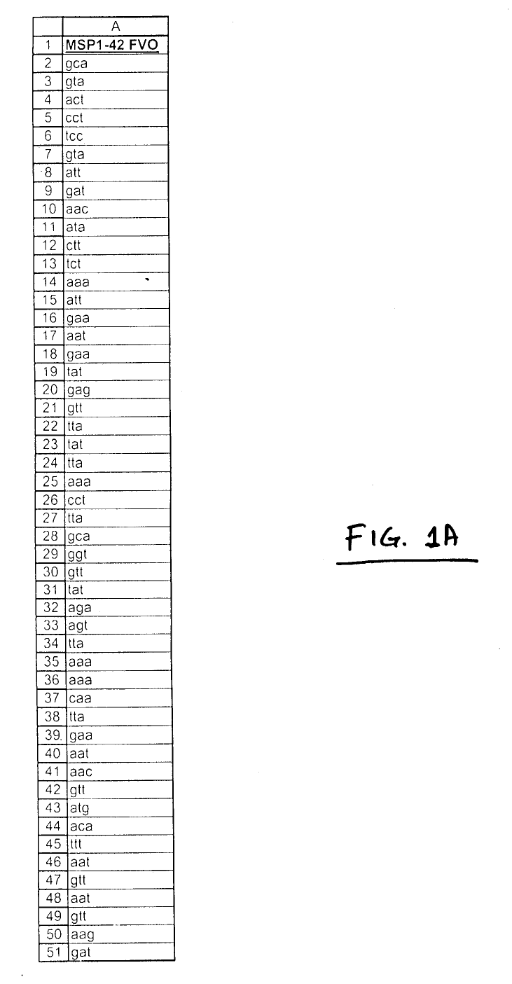

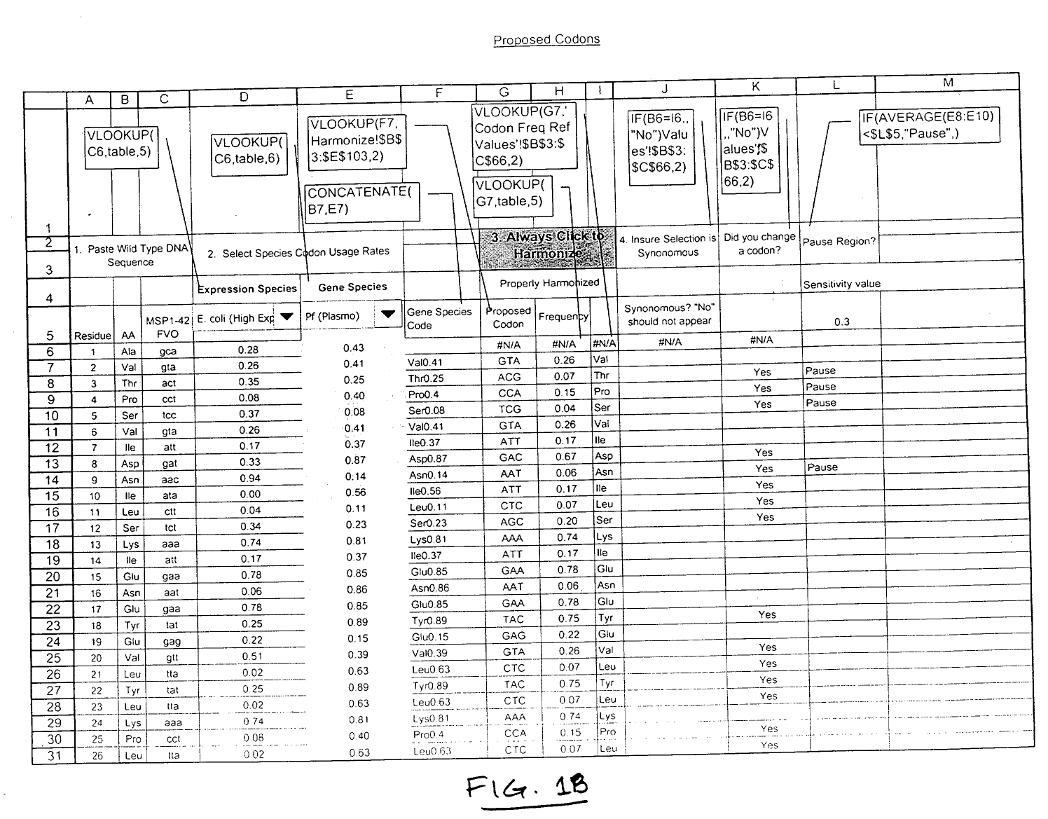

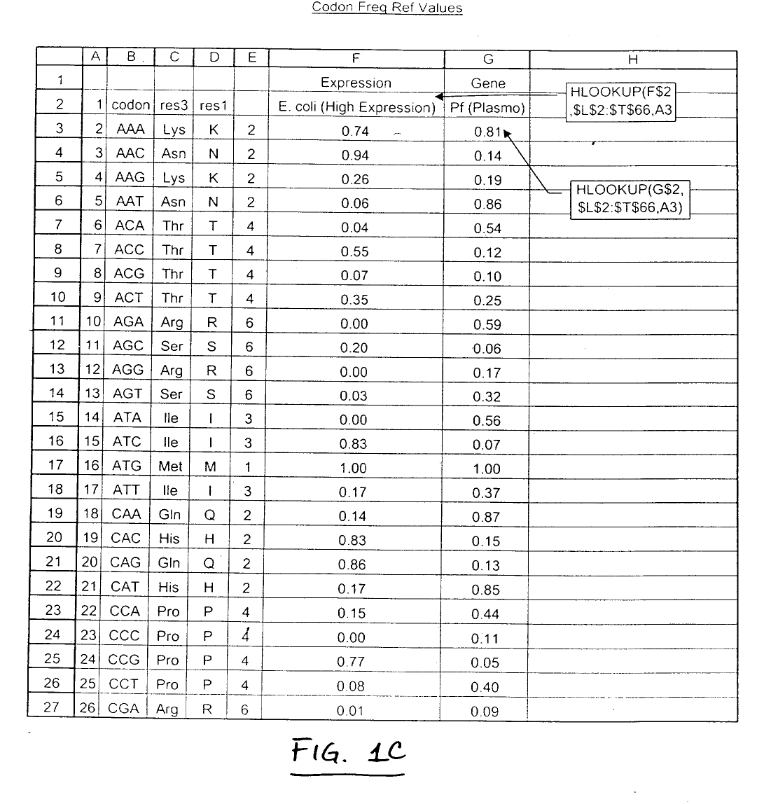

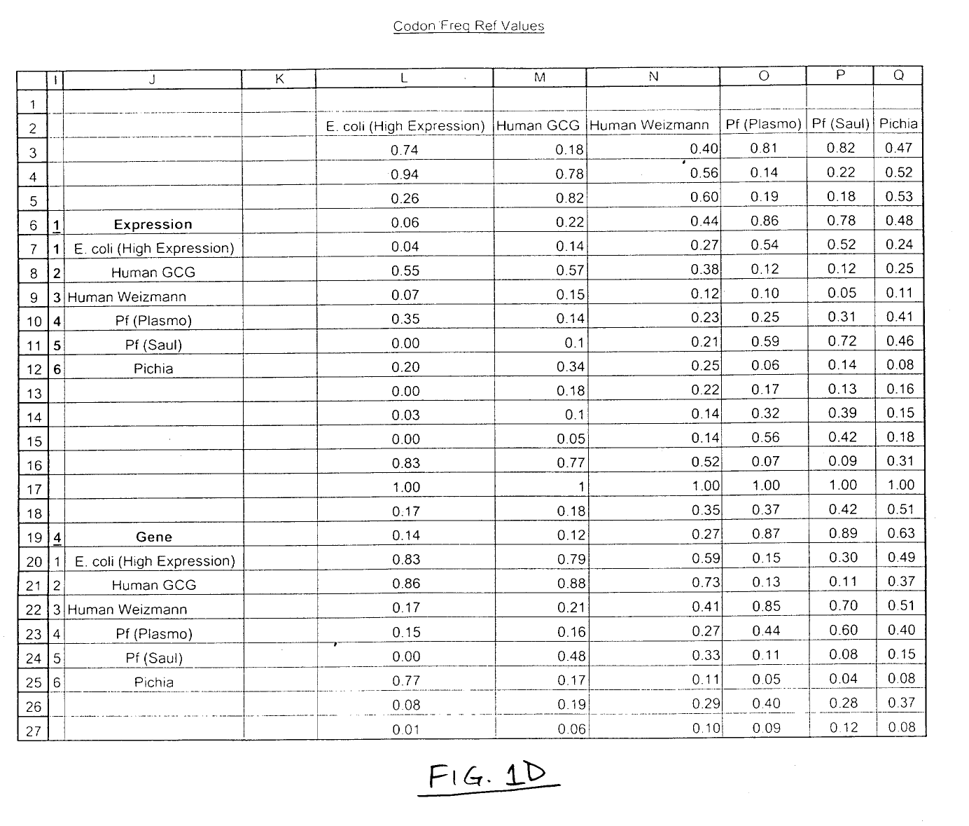

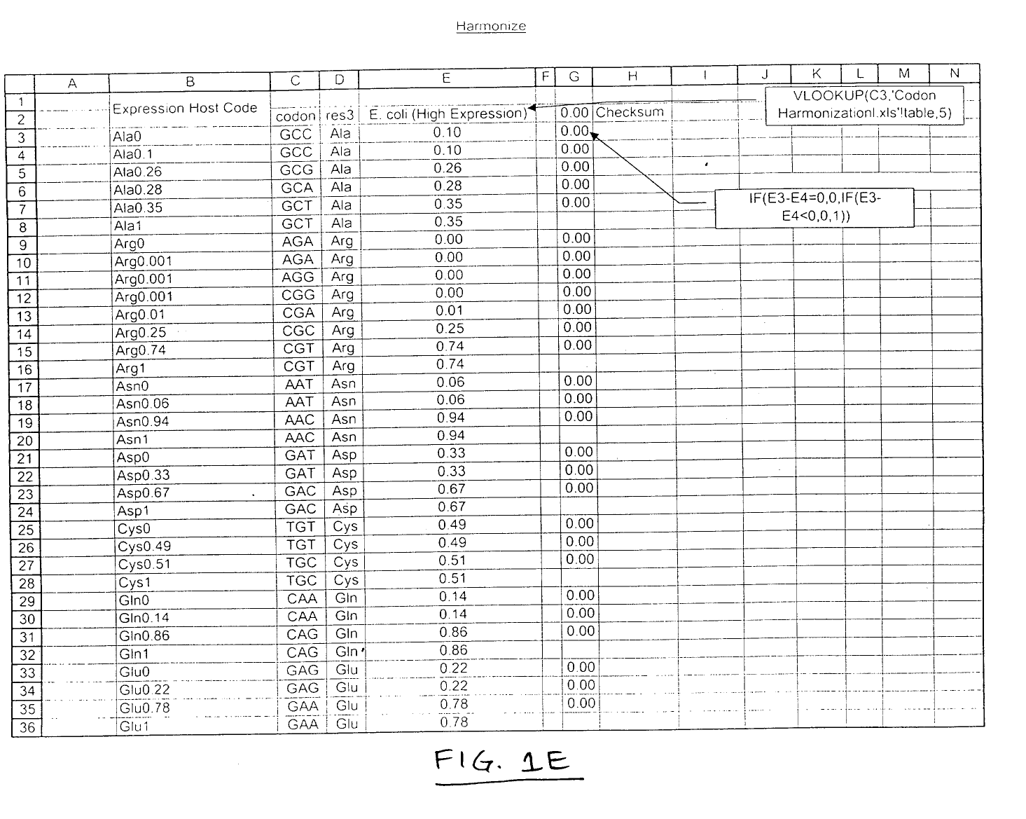

A title for the sequence is inserted manually into the first row of the target sequence (See FIG. 1A).

The sequence, including title is copied and pasted at Row 5, column C of the “Proposed Codons” spreadsheet (FIG. 1B). The amino acid corresponding to each codon is then printed next to the codon in Column B of the “Proposed Codons” spreadsheet. This is achieved by using the embedded Excel “vlookup” function to match the codon with its corresponding amino acid in Column C of the “Codon Frequency Reference Values” spreadsheet (FIG. 1C).

The name of the host (expression) species is selected from the dropdown box located in row 5 column D of the “Proposed Codons.” spreadsheet. This action finds that name in the range called “Host Species” on the “Codon Frequency Reference Values” spreadsheet, selects the number associated with that name and prints it to cell I19″ on that spreadsheet, where is it serves as an “index number.”.

This index number is used in conjunction with the embedded Excel “vlookup” function to report Host Species codon usaged frequencies in column F of the “Codon Frequency Reference Values” spreadsheet. The data in this column are also printed in Column D of the “Proposed Codons” spreadsheet. These data are reported for information only. They are not used further.

The name of the target gene species is selected from the dropdown box located in row 5 column E of the “Proposed Codons.” spreadsheet. This action finds that name in the range called “Gene Species” on the “Codon Frequency Reference Values” spreadsheet, selects the number associated with that name and prints it to cell I19″ on that spreadsheet, where is it serves as another “index number.”

This second index number is used in conjunction with the embedded Excel “vlookup” function to report Gene Species codon usage frequencies in column G of the “Codon Frequency Reference Values” spreadsheet. The data in this column are also printed in Column E of the “Proposed Codons” spreadsheet.

Two sets of unique names used to differentiate the various codons that can encode an amino acid by the usage frequency for that codon are created by using the embedded Excel “concatenate” function to combine the amino acid name with the frequency of usage of the codon for that amino acid. The first set of names (Gene Species Code) is reported in the “Proposed Codons” spreadsheet at Column F, and the second (Expression Host Code) is reported in the “Harmonize” spreadsheet (FIG. 1D) at Column B.

Clicking “3. Always Click to Harmonize” (macro 3) ranks the table in the “Harmonize” spreadsheet in ascending order according to “Expression Host Code” so that the “Gene Species Code” can be located correctly by using the “vlookup” function. When the Expression Species is changed the message “Error, click harmonize” will appear in at G4 in the “Proposed Codon” spreadsheet, until this macro is run.

Two outcomes result from the analysis are possible: 1. if the exact “gene species code” is found in the list of “expression host code” names (unlikely), the codon associated with the found “expression host code” (Column C of the Harmonize spreadsheet) is printed in Column G of the “Proposed Codon” spreadsheet, the usage frequency for that codon (Column F of the “Codon Frequency Reference Values” spreadsheet) is printed in Column H of the “Proposed Codon” spreadsheet, and the amino acid corresponding to that codon (Column C of the “Codon Frequency Reference Values” spreadsheet) is printed in Column H of the “Proposed Codon” spreadsheet. 2. if the exact “gene species code” is not found in the list of “expression host code” names (most likely), the codon associated with the next least frequently used codon described by the “expression host code” (Column C of the Harmonize spreadsheet) is printed in Column G of the “Proposed Codon” spreadsheet, the usage frequency for that codon (Column F of the “Codon Frequency Reference Values” spreadsheet) is printed in Column H of the “Proposed Codon” spreadsheet, and the amino acid corresponding to that codon (Column C of the “Codon Frequency Reference Values” spreadsheet) is printed in Column H of the “Proposed Codon” spreadsheet.

Column J is for quality control. The cells in this column compare the amino acid residues predicted after harmonization (Column I, “proposed codon” spreadsheet) with those of the foreign sequence (Column B). If “No” appears in any cell, the spreadsheet is corrupted and the calculation is not valid. If nothing is reported, the calculation is valid.

Column K is for information. The cells in this column compare the codons predicted after harmonization (Column G, “proposed codon” spreadsheet) with those of the foreign sequence (Column C) and report “yes” if a change is proposed.

Column L is another analysis tool, designed to identify “intradomain segments” or “pause regions” which should contain clusters of infrequently used codons. This tool examines the codon usage frequencies for the gene species by calculating a rolling average of the frequencies of usage of three consecutive codons found in Column E. Cell L5 sets the sensitivity of these calculations. Only average frequencies less than the “sensitivity value” are reported as “pause”. The larger this sensitivity value, the more pause sites are shown. This information is the first application of bioinformatics, other applications such as secondary protein structure predictions and mRNA secondary structure predictions can also be supplied. Additionally protein class (Henaut and Danchin: Analysis and Predictions from Escherichia coli sequences in: Escherichia coli and Salmonella, Vol. 2, Ch. 114:2047-2066, 1996, Neidhardt FC ed., ASM press, Washington, D.C.) and the changes in codon usage patterns associated with those classes will also represent additional important enhancements.

It should be understood that an existing DNA sequence can be used as the starting material and modified by standard mutagenesis methods that are known to those skilled in the art or a synthetic DNA sequence having the desired codons can be produced by known oligonucleotide synthesis, PCR amplification, and DNA ligation methods.

The frequency of codon usage in the wild-type DNA sequence is then compared to the frequency of codon usage in the host cell as shown in FIGS. 1A-E. Those codons present in the wild-type DNA sequence that have high frequency are changed to the synonymous host codons that have high frequency and the codons present in the wild-type DNA sequence that have low frequency are changed to the synonymous host codons which have low frequencies. It is understood that any changes to the DNA sequence always preserve the amino acid sequence of the wild-type protein. It is also a goal, through using bioinformatic analysis of data in the public domain-so called data mining- to deduce a basis for preferential harmonization of certain codons.

In one embodiment, the invention is related to designing a fully “harmonized” synthetic gene. A systematic bioinformatic analysis of secondary structure of the protein sequence to be expressed is carried out to correlate the utilization of infrequently-used codons with regions of protein structure (including but not limited to “turns” at the ends of coils, anti-parallel strands, extended beta sheets or helices and regions of disordered structure) that might necessarily require time to fold properly. Additional bioinformatic information such as protein sequence homology and secondary and/or tertiary structure homology may be “overlaid” to refine the anticipated need for inclusion or exclusion of such codons. There are many public software sources including the BLAST algorithm of NCBI, the EMBOSS package from the EMBL labs, and many programs that evaluate the three-dimensional structures of proteins deduced from x-ray crystallography or from NMR spectroscopy. By comparing the usage of low-frequency codons with these structural and structure-predicting programs over the gene information accumulated in public databases, it should be possible to gain prediction refinements and insights into the protein translation process.

In a further embodiment of the invention, consideration may be given to evaluating the classification of the protein that is the target for expression, by analogy to the several “classes” of protein (class I, class II and class III) in E. coli that utilizes codons differently. Thus far, the classes of genes are only categorized for E. coli and are based on their role in cell metabolism (class I) their propensity to be highly and continuously expressed (class II) or their apparent origin arising via lateral gene transfer (class III). The codon frequency tables for species other than E. coli use an aggregate of all protein coding regions to determine codon usage frequencies, yet it is clear that in E. coli, the codon usage differs greatly between these classes. In fact, the aggregate may not be the best criterion to generate the rules by which codons are harmonized. Such criteria, which probably can be established by protein sequence homology families, may be important. Those proteins which belong to different classes in other organisms/viruses may have preferred codon usages that are not simply those assumed from the aggregate sum of all codon usage in a particular organism. This type of bioinformatic information may add additional value by generating certain “rules” by which proteins have evolved and/or optimized their relative expression levels in specific biological contexts. Such rules may be employed in synthetic gene design and perhaps in development of altered paradigms for recombinant protein expression.

The resulting DNA sequence prepared according to the above description, whether by modifying an existing wild-type DNA sequence by mutagenesis or by the de novo chemical synthesis of a structural gene, is the preferred modified synthetic DNA sequence to be introduced into a host cell for enhanced expression and accumulation of the protein product in the cell.

The method of the present invention has applicability to any DNA sequence that is desired to be introduced into a host cell to provide protein product.

As will be described in more detail in the Examples to follow, the preferred modified synthetic DNA sequences were constructed by PCR mutagenesis which required the use of numerous primers. The primers were designed to introduce the desired codon changes into the starting DNA sequence. The preferred size for the primers is around 40-70 bases, but larger and smaller primers have been utilized. In most situations, a minimum of 5 to 8 base pairs of homology to the template DNA are maintained to insure proper hybridization of the primer to the template. Multiple rounds of mutagenesis were sometimes required to introduce all of the desired changes and to correct any unintended sequence changes as commonly occurs in mutagenesis. Also, in the Examples that follow, a totally synthetic DNA encoding the target protein sequence was synthesized by using long oligonucleotides of 55-65 nt, each with overlapping complementary ends, that were extended and amplified using PCR to generate modules of the gene. These modules were assembled by using ligation of appropriate restriction nuclease sites that are present in the designed sequence to yield the final synthetic gene product. It is to be understood that extensive sequencing analysis using standard and routine methodology on both the intermediate and final DNA sequences is necessary to assure that the precise DNA sequence as desired is obtained.

The DNA encoding the desired recombinant protein can be introduced into the cell in any suitable form including, the fragment alone, a linearized plasmid, a circular plasmid, a plasmid capable of replication, an episome, RNA, etc. Preferably, the gene is contained in a plasmid. In a particularly preferred embodiment, the plasmid is an expression vector. Individual expression vectors capable of expressing the genetic material can be produced using standard recombinant techniques. Please see e.g., Maniatis et al., 1985 Molecular Cloning: A Laboratory Manual or DNA Cloning, Vol. I and II (D. N. Glover, ed., 1985) for general cloning methods.

The following examples are illustrative in nature and are provided to better elucidate the practice of the present invention and are not to be interpreted in a limiting sense. Those skilled in the art will recognize that various modifications, truncations, additions or deletions, etc. can be made to the methods and DNA sequences described herein without departing from the spirit and scope of the present invention.

The following MATERIALS AND METHODS were used in the examples that follow.

Materials and Methods

Construction of Wild Type MSP1-42 (FVO)

Molecular cloning and bacterial transformations were performed as follows: MSP-1₄₂ fragment of FVO strain DNA was amplified by PCR from P. falciparum FVO genomic DNA by using the following primers:

| | | |
|---|---|---|
| FVO-PCR1; | 5′GGGTCGGTACCATGGCAGTAACTCCTTCCGTAATTGAT-3′ | (SEQ ID NO:1) |
| FVO-PCR2; | 5′GGATCAGATCCGGCCGCTTAACTGCAGAAAATACCATCGAAAAGTGGA-3′ | (SEQ ID NO:2) |

The primers contained restriction sites for restriction endonucleases, NcoI and NotI, respectively. The vector for expression of wild type sequence MSP1-42 (FVO), pET(AT)FVO, was prepared by digesting pET(AT)PfMSP-1₄₂ (3D7) (Angov et. al. (2003) Molec. Biochem. Parasitol; in press) and the MSP-1₄₂ PCR fragment, with NcoI and NotI. The digested DNA's were purified by agarose gel extraction (QIAEXII, Qiagen, Chatsworth, Calif.), ligated with T4 DNA ligase (Roche Biochemicals) and transformed into E. coli BL21 DE3 (F⁻ ompT hsdS_{B}(r_{B}⁻m_{B}⁻) gal dcm (DE3) [Invitrogen, Carlsbad, Calif.] (Maniatis). Two clones were sequenced and found to be identical in this region to Genbank Accession number, L20092. Analysis of soluble expression levels from this clone yielded poor product yields and therefore eliminated this construct from further development.

Construction of Single Pause Site Mutant Expression Vector: pET(AT)FVO.A

The initial approach to improve soluble protein expression was to apply the harmonization approach in a highly restricted way, which was to identify areas of the protein that were likely to represent intradomain segments owing to the presence of clusters of infrequently used codons in the wild type gene. This restricted approach was taken in order to minimize the cost of producing synthetic DNA. The analysis revealed a single codon within an intradomain segment near the N-terminus of the protein that might benefit from harmonization. To prepare the expression vector, pET(AT)FVO.A, two overlapping oligonucleotides from within the wild type MSP-1₄₂ (FVO) gene sequence were designed to introduce a single synonymous codon substitution at codon #158 (codon ATC was changed to ATA) by using PCR primer-directed mutagenesis.

EA3, 5′-TAAAAAATATATAAACGACAAAC-3′ (SEQ ID NO:3)

EA5, 5′-AAAAGGGAAGATATTTCTCATTT-3′ (SEQ ID NO:4) The base pair changes away from wild-type sequence are underscored. In the first amplification, the 5′ end of the wild type MSP1₄₂ (FVO) template was amplified by PCR with the sense external primer FVO-PCR1 and the anti-sense internal primer EA5. In the second amplification, the 3′ end of the wild type MSP1₄₂ (FVO) template was amplified by PCR with the sense internal primer EA3 and the anti-sense external primer, FVO-PCR2. The two PCR products were purified by gel extraction using QIAEX II, mixed (1:1) and were used as the template for a final amplification to produce full gene MSP-1₄₂ using flanking primers FVO-PCR1 and FVO-PCR2. The final clone was prepared by digesting the vector DNA, pET(AT)PfMSP-1₄₂ (3D7), and insert DNA, with NcoI and NotI, and ligating together. The final pET(AT)FVO.A plasmid encodes 17 non-MSP1 amino acids including a hexa-histidine tag at the N-terminus of P. falciparum FVO strain MSP-1₄₂ sequence.

Construction of “Initiation Complex” Harmonized MSP1-42 Expression Vector pET(K)FVO.B

The “initiation complex” harmonized MSP1-42 (FVO) clone was prepared by replacing the existing nucleotide sequence at the 5′-end of the MSP1-42 (FVO) gene sequence between restriction sites, KpnI and BspMI with annealed oligonucleotides that were designed to “harmonize” codon usage between P. falciparum usage and the E. coli host. To construct the “initiation complex” harmonized MSP1-42 (FVO), these two oligonucleotides pairs were synthesized, the sense strand, EA485-CDFVO,

| | |
|---|---|
| EA485-CDFVO | |
| 5′-CGCAGTTACTCCATCTGTTATTGATAATATTCTTTCTAAAA | (SEQ ID NO:5) |
| TTGAAAACGAATATGAGGTTTTATATTTAA3′ | |
| and | |
| EA493-CDFVO, | |
| 5′GGTTTTAATATAAAACCTCATATTCGTTTTCAATTTTAGAAAAGAATATTATC | (SEQ ID NO:6) |
| AATAACAGATGCAGTAACTGCGGTAC-3′ | |

The oligonucleotides were designed, as reverse complimentary strands with overhanging restriction sites at each end such that direct ligation into vector, pET(AT)FVO.A, would replace the existing 5′-nucleotide sequence between the KpnI and BspMI sites. The oligonucleotides were annealed by adding 100 nmole/ml of each oligonucleotide, in a buffer containing 0.01 M Tris-HCl, pH 7.5, 0.1 M NaCl, and 0.001M EDTA. The mixture was heated to greater than 95° C. for 10 minutes and then removed from the heat source and allowed to cool to room temperature. To prepare the vector DNA, pET(AT)FVO.A, the vector was first restriction digested with BspMI such that the DNA was only restricted at the BspMI site located within the MSP1-42(FVO) DNA and not at the second BspMI site, located in the vector DNA sequence. Linearized DNA, 7.8 kb, was separated by electrophoreses on agarose gels and then gel purified using QIAEX II. Extracted, purified linear BspMI pET(AT)FVO.A DNA was then digested with KpnI to release the “foreign” sequence initiation complex, ˜100 bp. The vector DNA, containing KpnI and BspMI restricted ends was gel purified and then ligated with the KpnI and BspMI annealed oligonucleotides. The ligated DNA was transformed into E. coli host, BL21 DE3 and plated onto ampicillin plates. Colonies were screened for the correct insert by restriction digestion with NcoI. Restriction positive clones were tested for expression using the laboratory's standard bacterial culture and expression methods. The novel MSP1-42 (FVO) “initiation complex” harmonized clone, expressed from plasmid pET(AT)FVO.B, demonstrated a 10-15 fold increase in levels of soluble protein as compared to the MSP1-42 (FVO) single pause site mutant, clone pET(AT)FVO.A. To generate the final expression vector, the MSP1-42 (FVO) “initiation complex” harmonized insert DNA from plasmid DNA, pET(AT)FVO.B, was subcloned into the newly constructed antibiotic resistance-gene modified pET vector, pET (K), by restriction digestion with BamHI and NotI. The final expression vector for expression of MSP1-42(FVO) “initiation complex” harmonized is pET(K)FVO.B.

Construction of the Full Gene Harmonized Expression Vector pET(K)FVO.C

To construct a synthetic gene for MSP1-42 (˜1100 nt), consecutive pairs of complementary oligonucleotides (each 50-60 nt , having 12-13 nt of unpaired sequence on the 5′ ends) were synthesized using fully harmonized sequence. Because the large size of the synthetic gene, four separate segments were created by using sequential PCR of the overlapping oligonucleotide pairs. The oligo pairs for PCR were selected so that the four segments could be joined by using three unique restriction enzyme sites (Hinc II, Bsrg I, Bst BI) present in the nucleotide sequence. To enable cloning into the pET(K) vector, an Nde I site was introduced just prior to the ATG initiation codon and tandem Not I and Xho I sites were included after the stop codon.

A series of PCR reactions yielded the four fragments. The first fragment begins with an Nde I site (before ATG codon) and ends with an Hinc II site. The second one starts with Hinc II and ends with a BsrG I site. The third one has BsrG I and Bst B I sites, and the last one had BstB I and Xho I sites (after the stop codon).

Each of the four fragments was generated separately and subcloned into a TA vector. In each instance, isolated transformants were selected and sequenced until a clone was identified as having the desired sequence and lacking mutations.

Each of the fragments was then purified from an agarose gel and ligated into a TA cloning vector, in sequence, by using T4 DNA ligase. For each step, competent host cells (TOP 10 supercompetent cells) were transformed with the ligation reaction and plated into antibiotic-selection plates and incubated at 37° C. Isolated colonies of transformants were grown to prepare plasmid DNA for agarose gel electrophoresis analysis. Several plasmids that appeared to contain insert were sequenced completely in order to select a clone without mutation. The final construct assembled from the four segments, pCR 2.1 -MSP(1-42), was purified in sufficient quantities to allow transfer to the final PET(K) expression vector.

Purified pCR 2.1-MSP(1-42) vector was digested with Nde I and Xho I and the insert purified on a 1% agarose gel. The purified 1.1 kbp fragment was ligated by using T4 DNA ligase into the PET(K) expression vector which had been digested with Nde I and Xho I and purified on 1% agarose gel. Competent host cells (TOP supercompetent cells) were transformed with the ligation reaction, plated into antibiotic-selection plates and incubated at 37° C. Isolated colonies of transformant were grown to prepare plasmid DNA for agarose gel electrophoresis analysis. Several plasmids that appeared to contain the final insert were sequenced in order to verify the integrity of the restriction sites.

Recombinant Protein Expression

For all constructions, E. coli B834 DE3 background cells were transformed with plasmids and were grown at 37° C. to an OD₆₀₀ of 0.5-0.8. The culture temperature was reduced from 37° C. to 25° C. prior to induction of protein expression with 0.1 mM IPTG. Induction was allowed to occur for 3.0 hours. At the end of the induction, cells were harvested by centrifugation at 27,666×g for 1 hr at 4° C. and the cell paste was stored at −80° C.

Partial Protein Purification for Comparison of Expression Levels

2-3 g cells were suspended in 20 ml 10 mM sodium phosphate, 50 mM NaCl, 10 mM imidazole, pH 6.2. The sample was lysed by using a microfluidizer and Tween 80 was added to a final concentration of 1%, and NaCl to a final concentration of 500 mM. The sample was stirred for 15 mi a 0-4° C., centrifuged for 30 min at 27,000 g at 0-4° C. and the supernate collected. The proteins were purified partially by chromatography on NI⁺² NTA Superflow (Qiagen, Chatsworth, Calif.). A 700 ul column was equilibrated with 0.01M sodium chloride, pH 6.2, 500 mM sodium chloride, 0.01 M imidazole (Ni-buffer) and 0.5% Tween 80. The sample was applied and the column washed with 10 ml of 10 mM sodium phosphate, pH 6.2, 75 mM sodium chloride, 0.02 M imidazole. The pH was the changed by washing with 10 ml 10 mM sodium phosphate buffer, pH 8.0, 75 mM sodium chloride, 0.02 M imidazole. The proteins were eluted in 3.5 ml of 10 mM sodium phosphate, pH 8.0, 75 mM sodium chloride, 160 mM imidazole and 0.2% Tween 80.

Partial Purification of E. coli Expressed Full Gene Harmonized MSP-1₄₂ (FVO) for Investigation of Solubility

Cell paste was lysed in buffer containing phosphate buffered saline, pH 7.4 containing 0.01 M imidazole and 50 U/ml benzonase. Following cell lyses by microfluidization, the lysate was either incubated in the presence or absence of the non-ionic detergent, Tween 80 (1.0%, v/v) on ice for 30 minutes with stirring, prior to centrifugation at 27,666×g for 1 hr at 4° C. This clarified lysate was centrifuged at 100,000 g for 1 hour to show that the protein is expressed in soluble form in the cell cytoplasm or it was applied to a Ni⁺² NTA superflow resin for partial purification.

SDS-PAGE and Immunoblotting

Proteins were separated by Tris-Glycine SDS-PAGE under non-reducing or reducing (10% 2-mercaptoethanol) conditions. Total protein was detection by Coomassie Brilliant Blue R-250 (Bio-Rad Laboratories, Hercules, Calif.) staining and immunoblotting are as previously described (3D7 manuscript). Nitrocellulose membranes were probed with either polyclonal mouse anti-FVO MSP-142 antibodies (a gift from Dr. Sanjai Kumar, FDA, Bethesda, Md.), polyclonal rabbit anti-E. coli antibodies (GSK) or mouse mAbs diluted into PBS, pH 7.4 containing 0.1% Tween 20. The mabs used for evaluation of proper epitope structure included 2.2 (McBride et al, 1987, Mol. Biochem. Parasitol., 23, 71-84; Hall et al, 1983, Mol. Biochem. Parasitol, 7, 247-65), 12.8 (McBride, 1987, supra; Blackman et al, 1990, J. Exp. Med., 172, 379-82), 7.5 (McBride, 1987, supra; Hall et al, 1983, supra), 12.10 (McBride, 1987, supra; Blackman et al, 1990, supra), 5.2 (Chang et al, 1988, Exp. Parasitol., 67, 1-11).

### EXAMPLE 1

Expression of LSA-NRC protein using “optimized” codon usage or “harmonized” codon usage in lsa-nrc gene constuction.

In this research, expression, purification and characterization of a recombinant P. falciparum LSA-1 gene construct, lsa-nrc, was undertaken with the aim of producing GMP grade protein for development as a pre-erythrocytic vaccine. The LSA-NRC protein contains the highly conserved N- and C- terminal regions and two 17 amino acid repeat units of the 3D7 sequence of the P. falciparum LSA-1 protein. Two distinct approaches were undertaken to improve the protein yield by genetically re-engineering the gene sequence from the original P. falciparum sequence. In the first approach the gene construct was designed using the highest frequency codons in E. coli, ie the gene was “optimized”. In the second approach, the gene construct was designed by “harmonizing” translation rates, as predicted by codon frequency tables, between P. falciparum and E. coli, to more closely match the translation rate in P. falciparum. An example of each approach is shown in the Table 2.

TABLE 2

| | Usage rate | E. coil | Codon | | Codon |
|---|---|---|---|---|---|
| Original P. | of original | abundance | usage rate of | Harmonize | usage rate of |
| falciparum | codons in | optimized | Isa-nrc/E in | d Isa-nrc/H | Isa-nrc/H in |
| codons | P. falciparum | codons | E. coil | codons | E. coil |
| AAC | 0.14 | AAC | 0.94 | AAT | 0.06 |
| TTG | 0.14 | CTG | 0.83 | CTC | 0.07 |
| AGA | 0.59 | CGT | 0.74 | CGC | 0.25 |

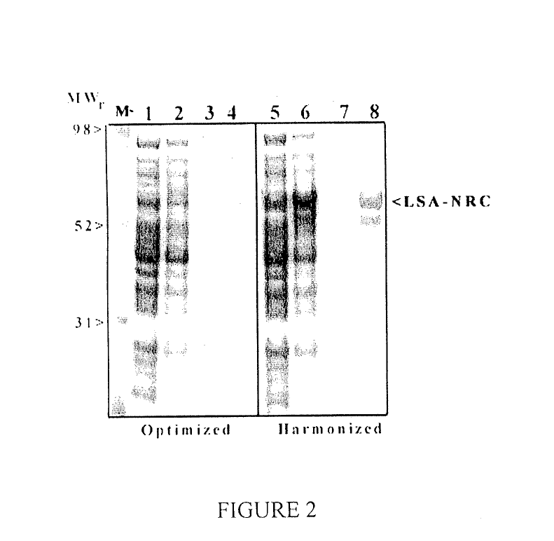

Making an lsa-nrc gene for heterologous expression by “harmonizing” translation rates (lsa-nrc/H) was more effective than using highest frequency E. coli (lsa-nrc/E) codons. It provided for the high-level expression of soluble protein. See FIG. 2.

### EXAMPLE 2

Coomassie Blue stained SDS-PAGE for Partially Purified Wild type MSP1-42 (FVO) vs. Single Site pause mutant (FMP003).

We found that the levels of soluble MSP1-42 (FVO) protein obtained following induction of BL21 DE3 cells expressing the wild type gene sequence, pET(AT)FVO was negligible and insufficient to advance for further process development. Rather than simply changing to a new expression system, such a Pichia, or baculovirus, we chose to try to fix this problem owing to the advantages that E. coli offers, especially with respect to expression of non-glycosylated protein. Our initial thinking was that it might be important to preserve ribosomal pausing at certain times during translation to allow for protein folding. We thought that we might achieve this by analyzing the target gene to reveal clusters of low abundance condons and changing those codons if necessary (harmonizing) so that they would be low abundance in the expression host (in this case E. coli). For the first approach for codon harmonization, we used, as reference materials, codon frequency tables for P. falciparum (Saul A & Battistutta D. Codon usage in Plasmodium falciparum. Mol Biochem Parasitol 1988;27:35-42.) and E. coli (Data Reference Set, Volume 3: Data Files, Genetics Computer Group, Sequence Analysis Software Package). We evaluated consecutive codons as rolling triplets along the range of amino acids of interest, paying special attention to the patterns associated with domain segments, which separate minimal domain structures, i.e. alpha helices, beta pleated sheets. Within interdomain segments, the amino acid content is restricted to about half of the common amino acids and their corresponding codons tend to be used infrequently, indicating that translation proceeds slowly in these regions. This slowdown in translation within interdomain segments may allow nascent protein to complete the folding of one domain prior to initiating synthesis of the next.

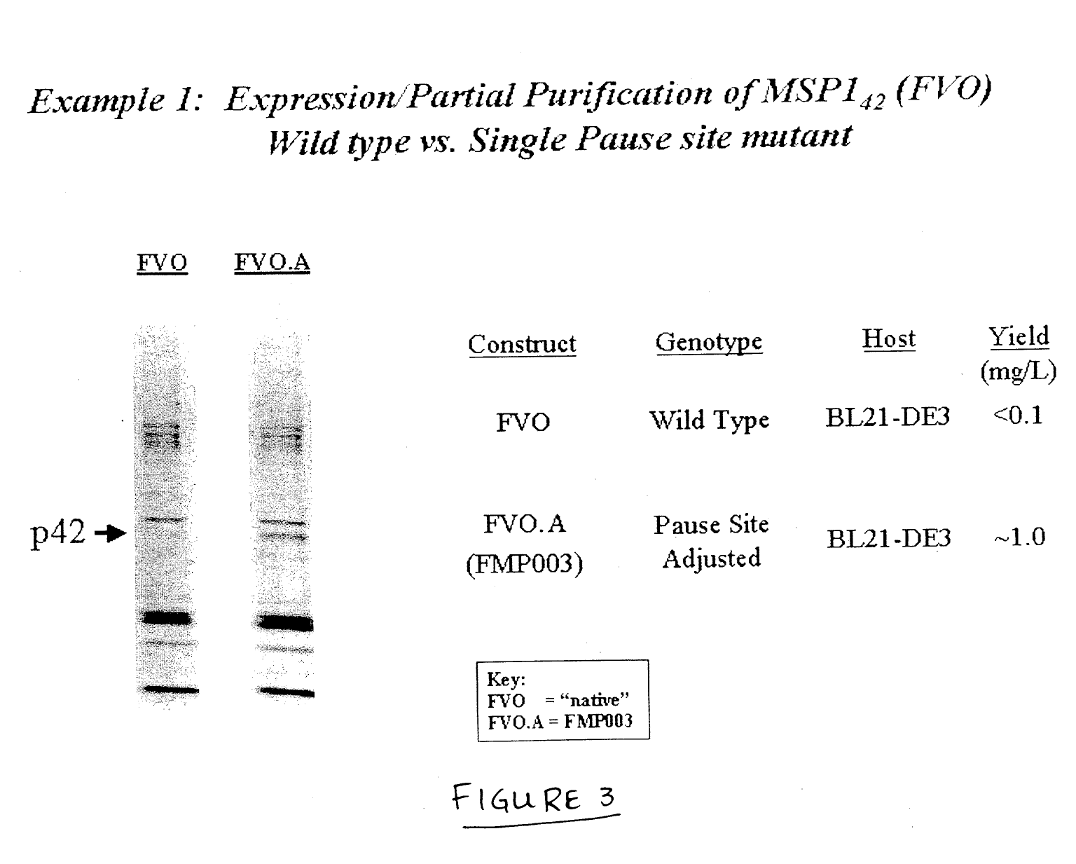

Using this method we predicted putative translation pause sites (low frequency used codons in P. falciparum) and we identified a single amino acid substitution within the translated sequence, #158, which required harmonization for low frequency expression in E. coli. The Coomassie Blue stained gels shown in FIG. 3 compares partially purified wild type vs. single pause site mutant MSP1-42 (FVO), FMP003. The relative increase in soluble MSP1-42 expression is approximately 10 fold above wild type. At that time we recognized that “fully harmonizing” a gene might be the best strategy; we took this initial “limited” approach owing to the expense associated with making synthetic genes.

### EXAMPLE 3

Coomassie Blue stained SDS-PAGE on Partially Purified MSP1-42 (FVO) (Wild type vs. Single Site pause mutant (FMP003) vs. Initiation Complex harmonized (FMP007))

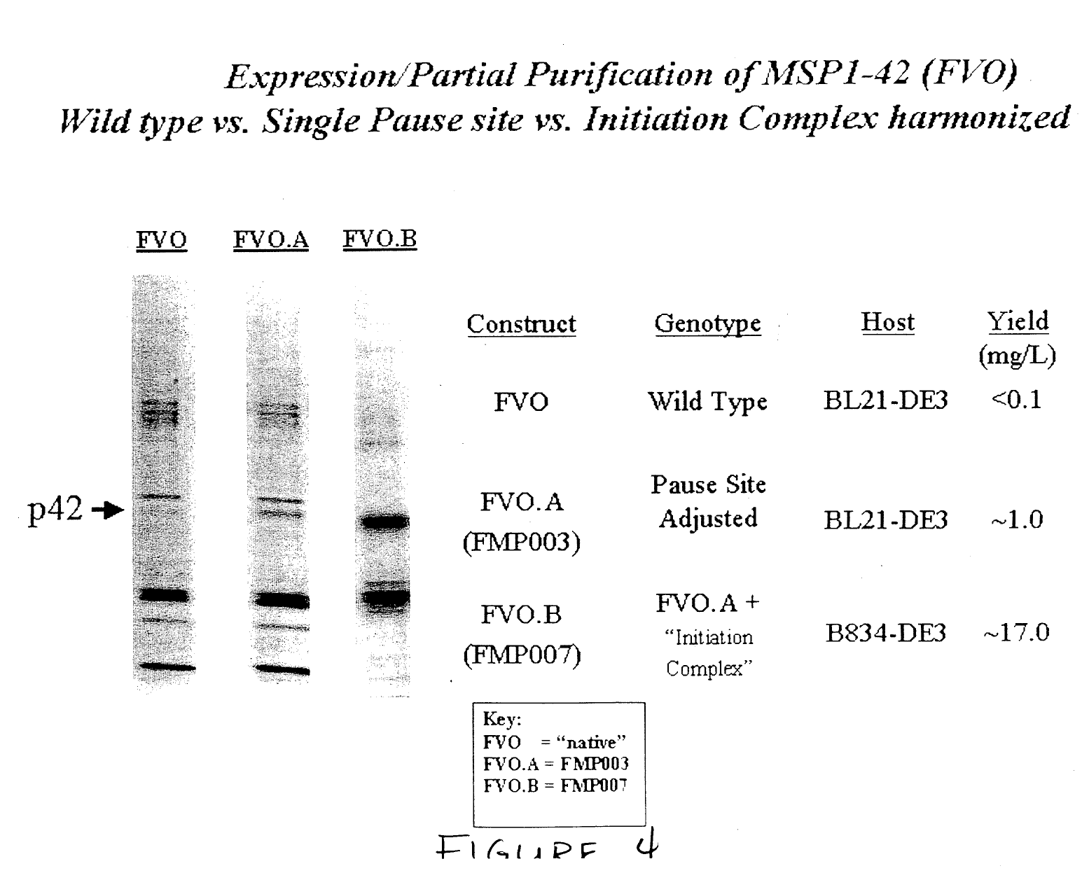

While the FMP003 product was estimated to yield approximately 10 fold more soluble MSP1-42 than wild type sequence, the final product yield, at 1 mg/L, was still insufficient for advanced development where target product yields are in the range of 100 mg/L. Therefore, for the second approach, E. coli codons were harmonized to P. falciparum codons with the objective of preserving high and low usage rates in the region of the initiation complex. A hypothesis is that stabilizing the interaction of the ribosome on the initiation complex might lead to increased levels of translation, or that translation from a properly harmonized initiation complex might allow for the initiation of proper protein folding. Again, using existing codon frequency tables referred to above, we applied the same process more broadly to reveal all codons in the “initiation complex” region that were mismatched for codon usage frequency between the target gene and the expression host. Five synonymous codon replacements were made and resulted in an additional 10-15 fold increase in soluble product when compared to FMP003. The estimated product yield for FMP007 is 15 mg/L based on small-scale chromatography. The levels of final product produced are substantially above the wild type MSP1-42 and the FMP003 product (FIG. 4). Given the improvement in yield of FMP007 compared with FMP003, we decided to try a fully harmonized gene. This decision was supported by our results from the full gene harmonization for the malaria antigen, LSA-NRC, which lead to bacterial expression levels in the range of 30-50% of the total protein from a cell lysate, all of which was soluble in the host cell cytoplasm.

### EXAMPLE 4

Coomassie Blue stained SDS-PAGE & Western blot Analysis of lysates from bacteria expressing FMP003, FMP007, or full gene harmonized.

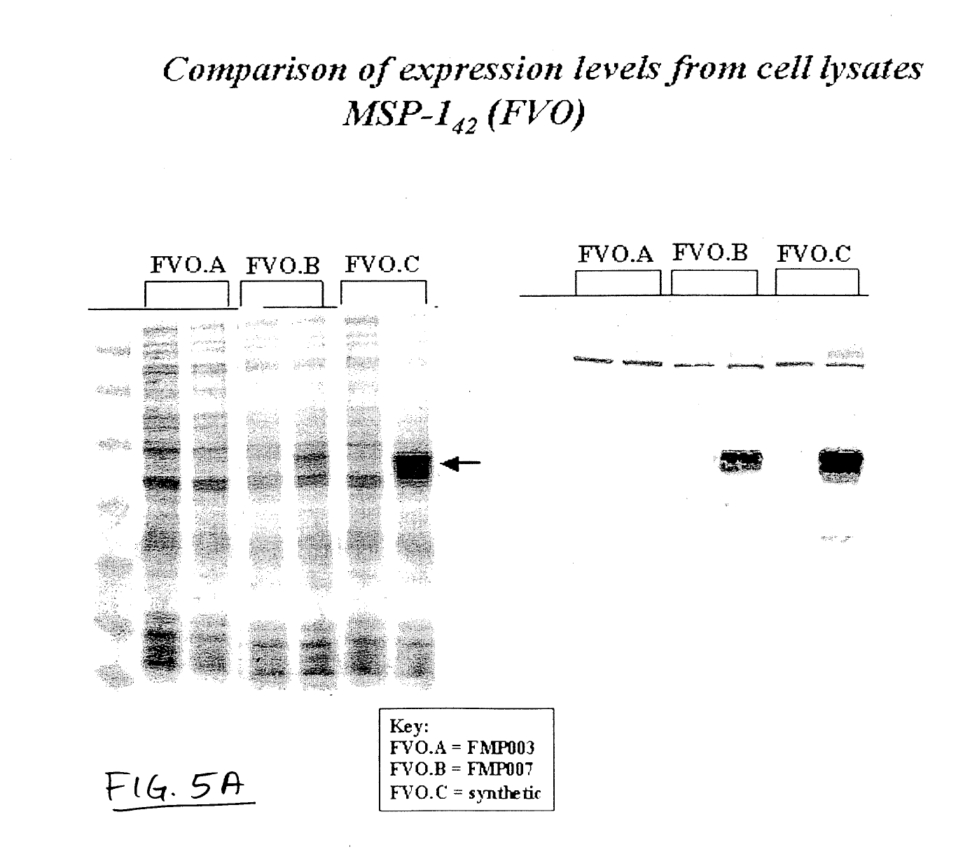

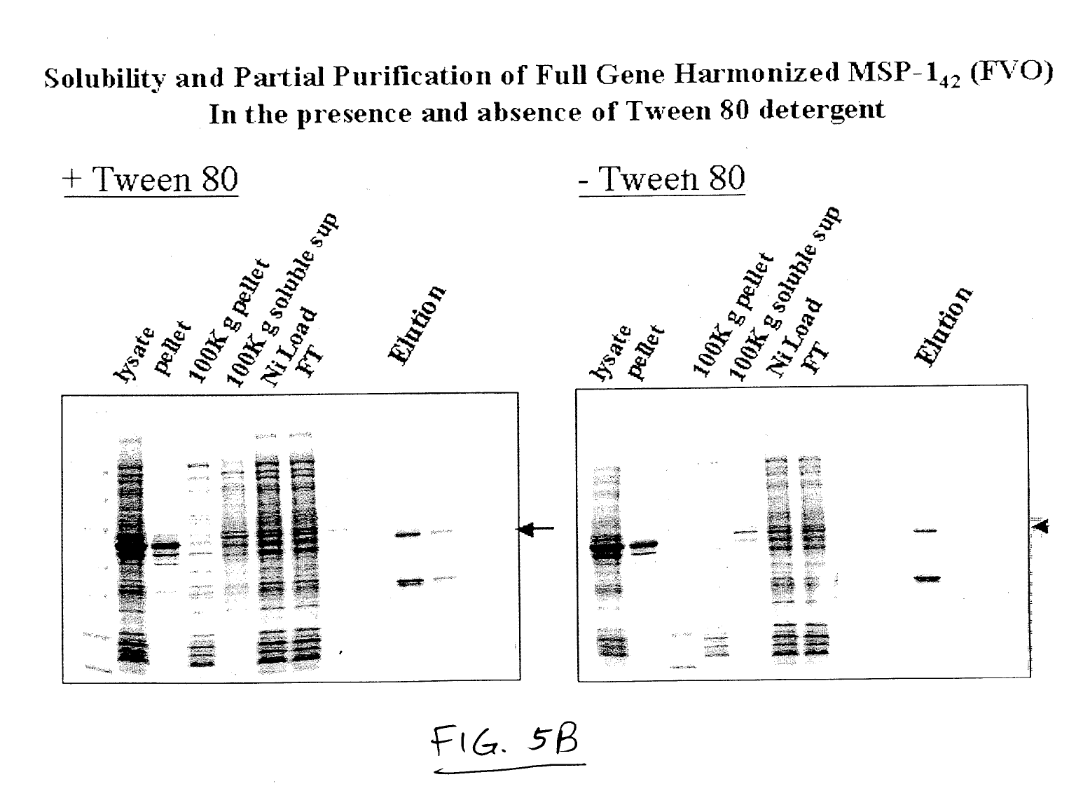

For the final approach, E. coli codons were harmonized to P. falciparum codons with the objective of preserving all high and low codon usage rates throughout the gene sequence. This effort resulted in additional 10-fold increase in the yield of protein from the fully harmonized gene over that of FMP007 (FIG. 5A) and at least half of the protein was soluble in the host cell cytoplasm (FIG. 5B).

What is claimed is:

1. A method for designing a synthetic gene for optimal expression, in a host cell, of a foreign protein encoded by a foreign gene comprising
(i) determining the frequency of codon usage of foreign gene coding sequence, and
(ii) substituting codons in the foreign gene coding sequence with codons of similar frequency from the host cell which code for the same amino acid.

2. A synthetic DNA sequence prepared according to claim 1.

3. A host cell transformed with the synthetic DNA sequence of claim 2.

4. The method of claim 1 wherein said host cell is prokaryotic.

5. The method of claim 4 wherein said prokaryotic cell is E. coli.

6. The method of claim 1 wherein said foreign gene is from P. falciparum.

7. The method of claim 4 wherein said foreign gene is P. falciparum.

8. A method for identifying codons in a foreign gene which need to be harmonized with codons of a host, the method comprising: providing a database including codons-usage frequency for a plurality of types of organisms; displaying a list of types of organisms;receiving a user's selection of foreign gene codons; determining degree of difference in codon-usage frequency between the selected host and foreign gene for similar amino acid codons; and displaying results of said determination wherein codons of similar frequency for a similar amino acid are recommended for harmonization with host codons.

9. A synthetic gene harmonized using the method of claim 8.

10. A computer system comprising: a database of codon-usage frequencies for a plurality of types of organisms; and a user interface capable of receiving a selection of foreign gene codons for comparison of codon-usage frequency; and displaying the results of said comparison.

11. A computer program product comprising a computer-usable medium having computer-readable program code embodied thereon relating to a database including codon-usage frequencies for a plurality of types of organisms, the computer program product comprising computer-readable program code for effecting the following steps within a computing system: providing an interface for displaying at least one list of said codon-usage frequencies; receiving via said interface a user's selection of one or more codons for foreign genes; comparing codon-usage frequencies from an organism chosen from said list with codon-usage frequency from foreign gene; determining if harmonization of said foreign codon is recommended; and displaying the results of said determination.


SEQUENCE LISTING (6 sequences)

SEQ ID NO: 1 - DNA, 38 bp, Artificial sequence
  designed primer
gggtcggtaccatggcagtaactccttccgtaattgat

SEQ ID NO: 2 - DNA, 48 bp, Artificial sequence
  designed primer
ggatcagatgcggccgcttaactgcagaaaataccatcgaaaagtgga

SEQ ID NO: 3 - DNA, 23 bp, Artificial sequence
  designed primer
taaaaaatatataaacgacaaac

SEQ ID NO: 4 - DNA, 23 bp, Artificial sequence
  designed primer
aaaagggaagatatttctcattt

SEQ ID NO: 5 - DNA, 71 bp, Artificial sequence
  designed primer
cgcagttactccatctgttattgataatattctttctaaaattgaaaacgaatatgaggttttatatttaa

SEQ ID NO: 6 - DNA, 79 bp, Artificial sequence
  designed primer
ggttttaaatataaaacctcatattcgttttcaattttagaaagaatattatcaataacagatggagtaactgcggtac